(19) United States
(12) Patent Application Publication
(10) Pub. No.: US 2025/0212844 A1
(43) Pub. Date: Jul. 3, 2025

(54) Quadruped Lameness Detection using Machine Learning Models

(71) Applicant: Bay West Veterinary Surgery, Inc., Woodside, CA (US)

(72) Inventor: Dean Filipowicz, Woodside, CA (US)

(21) Appl. No.: 18/402,060

(22) Filed: Jan. 2, 2024

(51) Int. Cl. A01K 29/00; A61D 99/00; G16H 50/20

(52) U.S. Cl. CPC A01K 29/005; A61D 99/00; G16H 50/20

(57) ABSTRACT

Systems and methods for detecting lameness in a limb of an animal using a machine learning model are described herein. In an implementation, a system receives first data measurements from a sensor device attached to the animal, where the first data measurements represent measurements associated with gait of an animal. Start and stop times are determined for movement events identified from the first data and the movement events are stored. A first machine learning model is used to generate levels of gait asymmetry using the movement events as input. The first machine learning model is a model trained to predicts levels of gait asymmetry based on movement data. Upon generating the levels of gait asymmetry, one or more treatment recommendations are generated based on the levels of gait asymmetry. The one or more treatment recommendations and levels of gait asymmetry are displayed on a client computing device.

### FIELD OF THE DISCLOSURE

The present disclosure generally relates to data processing systems using wireless networks and wearable sensors. More specifically, the disclosure relates to computer systems linked to sensors for the purpose of detecting deviations from symmetry in the use of limbs of an animal to identify and chronicle changes in lameness.

### BACKGROUND

The approaches described in this section are approaches that could be pursued, but not necessarily approaches that have been previously conceived or pursued. Therefore, unless otherwise indicated, it should not be assumed that any of the approaches described in this section qualify as prior art merely by virtue of their inclusion in this section.

Lameness is a term used to describe an animal's change in gait in response to pain in a limb or in response to a mechanical restriction on movement. Lameness may be caused by musculoskeletal injuries and/or disease in quadruped animals such as domestic dogs and cats. For example, various kinds of ligament and tendon injuries, degenerative joint diseases, insidious bone diseases and other ailments may be manifested in lameness. In this context, lameness refers to the inability of an animal to walk in a normal fashion or to bear full weight on all the limbs in the manner typically observed or experienced in a healthy condition.

However, at present, detecting lameness usually is performed in ways that have significant drawbacks. A first approach involves visual observation and estimation of gait or movement followed by evaluation of an animal's response to stimulus. This approach involves subjective individual analysis that is prone to error and usually only can identify large changes in lameness and/or load bearing starting at higher grades of lameness. It is inherently non-scientific and imprecise.

Another approach involves using a stationary force plate or force pad, alone or in conjunction with secondary quantitative analysis such as goniometry and/or limb circumference measurements. Kinetic analysis based on data from a force plate can be used to generate force time curves in three dimensions as well as values for peak vertical force (PVF), defined as the maximum force in the vertical dimension. These data can be used to evaluate craniocaudal direction in the sense of propulsion and braking, and mediolateral direction or turning. However, the data gathering apparatus is costly, esoteric in nature, and requires detailed training of the subject animal to ensure that load cells are struck and velocity is constant. Furthermore, the apparatus does not measure stride length or duration and is difficult to set up.

Force pads can be used for temporospatial gait analysis to indicate the duration of phases of stride, stride velocity, stride length, step length and a total pressure index. Duration of phases of stride refers to distinguishing stance versus swing. Stride length can be measured as the distance between paw strikes of the same limb. Step length can comprise the distance from an aspect of paw strike of one limb to the same aspect of the strike of a paw of a contralateral limb. The total pressure index may be the sum of peak pressure values recorded on the pad from each activated sensor by a paw during mat contact; it is related but not equal to peak vertical force. Force pad apparatus also is costly, requires a large physical space, and is esoteric. Additional forces present during ambulation can interfere with and complicate measurement; examples include forces to change speed, direction or maintain balance. Further, the apparatus only measures total ground reaction forces and not component vectors.

Kinematic gait analysis typically involves using markers affixed to joints and recording the movement of the markers using a video camera, followed by secondary quantitative analysis such as goniometry and/or limb circumference measurements. This approach is costly, difficult or impractical to perform with some animals, and produces data that is not easily repeatable. There can be random error in the data due to skin movement, and creating reference values is not supported.

Based on these deficiencies in current practice, there is a need for an improved easily available apparatus for reliably and precisely quantitating lameness in animals.

### SUMMARY

The appended claims may serve as a summary of the invention.

### BRIEF DESCRIPTION OF THE DRAWINGS

In the drawings:

FIG. 1A illustrates an example lameness assessment system, according to an implementation.

FIG. 1B illustrates internal elements of an example sensor device of FIG. 1A, according to an implementation.

FIG. 1C depicts an example of a server device implemented to execute one or more machine learning models for predicting gait asymmetry of an animal and for predicting lameness severity and treatment recommendations based on observed sensor data from one or more sensor devices associated with the animal, according to an implementation.

FIG. 2 depicts a flowchart for automatically detecting lameness in an animal based on movement events and generating a recommended treatment and management options, according to an implementation.

FIG. 3 illustrates an example graphical user interface (GUI) with example visualization of lameness data.

FIG. 4 depicts a graphical user interface depicting comparisons of data for the limbs of an animal.

FIG. 5 illustrates an example computer system with which implementations may be implemented.

### DETAILED DESCRIPTION

In the following description, for the purposes of explanation, numerous specific details are set forth in order to provide a thorough understanding of the present invention. It will be apparent, however, that the present invention may be practiced without these specific details. In other instances, well-known structures and devices are shown in block diagram form in order to avoid unnecessarily obscuring the present invention.

### General Overview

According to an implementation, a lameness measurement system comprises a plurality of wearable sensor devices, each comprising a sensor and a short-distance wireless data transmitter, a computing device having a storage device holding an application program that is programmed to obtain sensor data from the sensors and communicate the sensor data to one or more machine learning models to calculate gait asymmetry of an animal and to provide treatment recommendations for the animal's detected lameness. In some implementations, the system further comprises a wireless data relay device that is configured to receive sensor data from the sensors via a first wireless networking protocol, to transform the sensor data into electronic messages, optionally with analysis, compression or other operations, and to transmit the transformed sensor data via a second wireless networking protocol to the computing device. In some implementations, the wireless data relay device is miniature and may be affixed to a wearable device, such as a collar of the animal, a part of a harness, or any other wearable. In some implementations, the computing device is a mobile computing device and the application program is implemented as a compact program or mobile app.

In an implementation, the system includes a server device that implements one or more machine learning models programmed to detect acute lameness and/or lameness over time in a quadruped animal and generate treatment recommendations for the detected lameness. In an implementation, the machine learning models are trained using a corpus of observed data associated with gait of various animals covering multiple observations over various periods of time. The corpus of observed data also includes physical characteristics and medical history associated with the various animals. In some implementations, the one or more machine learning models of the system are programmed to detect lameness in previously quadruped animals. For example, the system may be trained to detect lameness in an amputee animal, where the system determines whether there is a change in gait in the remaining limbs of the amputee animal over a period of time.

In some implementations, the data relay device or one of the sensor devices further comprises a global positioning system (GPS) transceiver that is configured or programmed to obtain GPS radio signals from earth orbiting satellites and to compute a geo-spatial position or location position based upon the GPS signals. In such an implementation, the GPS transceiver may be programmed or configured to accept calls, polls, or other signals from the data relay device, sensor devices or computing device to report then-current geo-location data values such as a pair of latitude-longitude (lat-long) values. These values also may be periodically transmitted without a request or poll signal according to a schedule, such as once per second.

In an implementation, the lameness measurement system receives at data relay device, from one or more sensor devices, first data representative of measurements associated with gait of the animal. The first data may include force or pressure data from paw strikes, kinematic data collected from various sensor devices equipped with accelerometers, video and audio captured from recording devices, and any other sensor data. The lameness measurement system determines start and stop times for movement events, where a movement events represent instances of movement or non-movement for an animal, and stores the movement events in persistent storage.

In an implementation, the lameness measurement system uses a first machine learning model to generate levels of gait asymmetry for the animal, using the movement events as input. The first machine learning model may represent an asymmetry prediction machine learning model that has been trained to detect levels of asymmetry in gait based on various types of sensor data. For example, the asymmetry prediction machine learning model may determine, previously undetectable levels of gait asymmetry based on a combination of differences between observed kinematic movements of limbs of the animal, differences in paw strike force/pressure values, and any other observations of the animal during movement. The asymmetry prediction machine learning model generates levels of gait asymmetry for the animal.

In an implementation, the lameness measurement system determines, based on the levels of gait asymmetry, one or more recommendations for management of treatment for the animal. For instance, the treatment may identify a potential injury, such as a torn ligament, and may suggest professional treatment or surgery for the animal. Additionally, the lameness measurement system may suggest sets of exercises to perform to improve the lameness detected, or sets of activities to avoid based on the lameness detected and potential injury identified.

Implementations will find utility with surgeons, neurologists, oncologists, rehabilitation providers, and general practitioners, as well as handlers or owners of working animals, owners of animals that have recognized injuries and/or disease, owners of animals that are at risk of injury or disease based on breed or history, and owners of animals that are difficult to handle or transport. Additionally, implementations may find utility for assessing potential liability for insurance companies, pharmaceutical companies, animal breeders, and any other service associated with activities that may assess risk to the health of the animal, such as dog walkers or animal boarding facilities. Implementations may also find utility when methods for performing orthopedic exams are inconsistent or inaccurate, such as with cats or small dogs which compensate better for subtle lameness, making the lameness more difficult to visualize or identify. Implementations can be produced at reasonable cost and are configured for ease of use and ease of understanding. Implementations provide precise and repeatable data with the introduction of no significant systemic error.

Implementations are contemplated for use with small quadruped animals, including, but not limited to, breeds of dogs and cats.

### Structural Overview

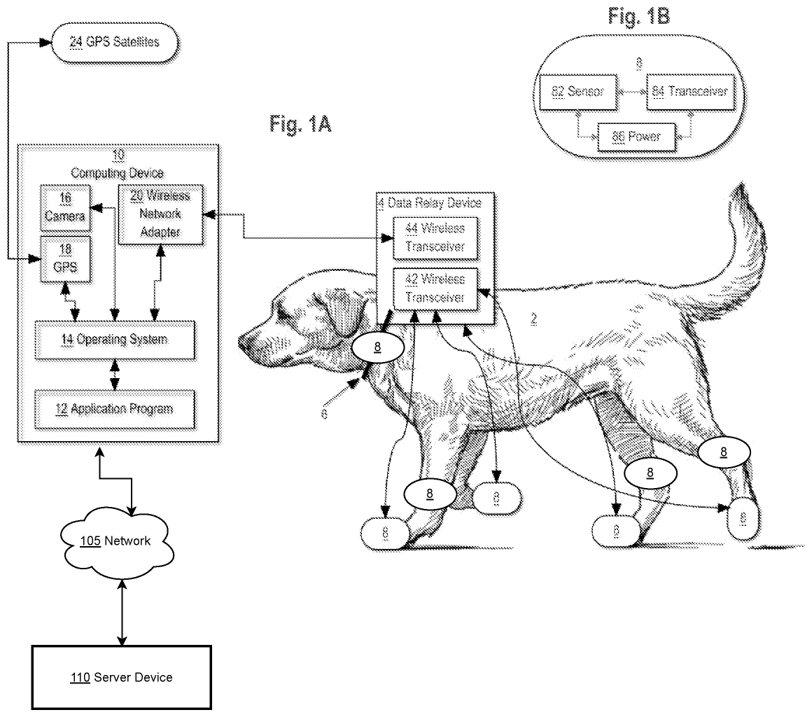

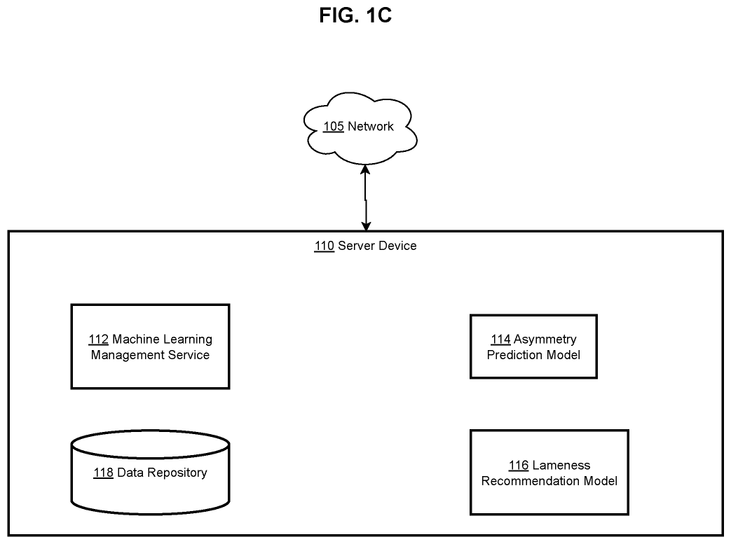

FIG. 1A illustrates an example lameness assessment system, according to one implementation. Quadruped animal 2 is fitted with a data relay device 4 that is integrated with, affixed to, or mounted on collar 6. Animal 2 depicts a dog, having four (4) paws. Animal 2 is shown fitted with a plurality of sensor devices 8 each comprising a sensor and a wireless transceiver that is capable of wireless data communication with a first wireless transceiver 42 of the data relay device 4. While methods are described with respect to four legged animals, implementations may be applied to animals with less legs, such as amputees, in order to assess possible incipient pathology. Sensor devices 8 pictured in FIG. 1A may include footwear, strapped bands, collar-based sensors, and any other type of sensor device that may be affixed, either temporarily or permanently, to the animal 2. In one example, sensor devices 8, may be footwear, which may include a set of boots or socks designed to fit over the animal's 2 paws. In another example, sensor devices 8 may represent sensors attached to an assortment of straps that may be strapped around animal 2's limbs. In yet another example, sensor devices 8 may represent a sensor device that is affixed on or within the collar 6. In some implementations, sensor devices 8 may also include sensors that are not affixed to animal 2, such as a video capture device and/or an audio capture device. The video and/or audio devices may be configured to capture the movement of animal 2 during an activity session. For instance, a video camera may be used to capture movement of animal 2. Additionally, a microphone may represent an audio capture device, where the microphone may capture acoustic sounds that may be made when animal 2 walks or trots across a hard floor, such as a hardwood or cobblestone floor.

Computing device 10 comprises an application program 12, operating system 14, camera 16, GPS transceiver 18 and wireless network adapter 20. Application program 12 executes on or is hosted by the computing device 10 and is configured or programmed with stored program instructions which when executed cause performing the functions, operations and process steps that are further described in other sections herein. Camera 16 may be integrated into the computing device 10 or arranged as a peripheral that is coupled to the computing device using a parallel data or serial data I/O interface. In one implementation, camera 16 is controlled by subroutines organized as primitive services of the operating system 14 and that are callable using programmatic calls using a defined API or operating system call facility. GPS transceiver 18 is configured or programmed to communicate wirelessly by radio with GPS satellites 24 in orbital positions around the earth and to calculate a geo-location position of the computing device 10 in terms of latitude, longitude values based on wireless signals from the satellites. In some implementations, GPS transceiver 18 may be configured or programmed to be compatible with various current and future iterations of GPS technology, including, but not limited to, Navigation Indian Constellation (NAVIC) technologies and any other emerging GPS technologies. The wireless network adapter 20 is communicatively coupled to the data relay device 4 via a second wireless transceiver 44 in the data relay device. In some implementations, computing device 10 may include additional features not depicted in FIG. 1, such as a microphone that may be used to capture audio from the animal.

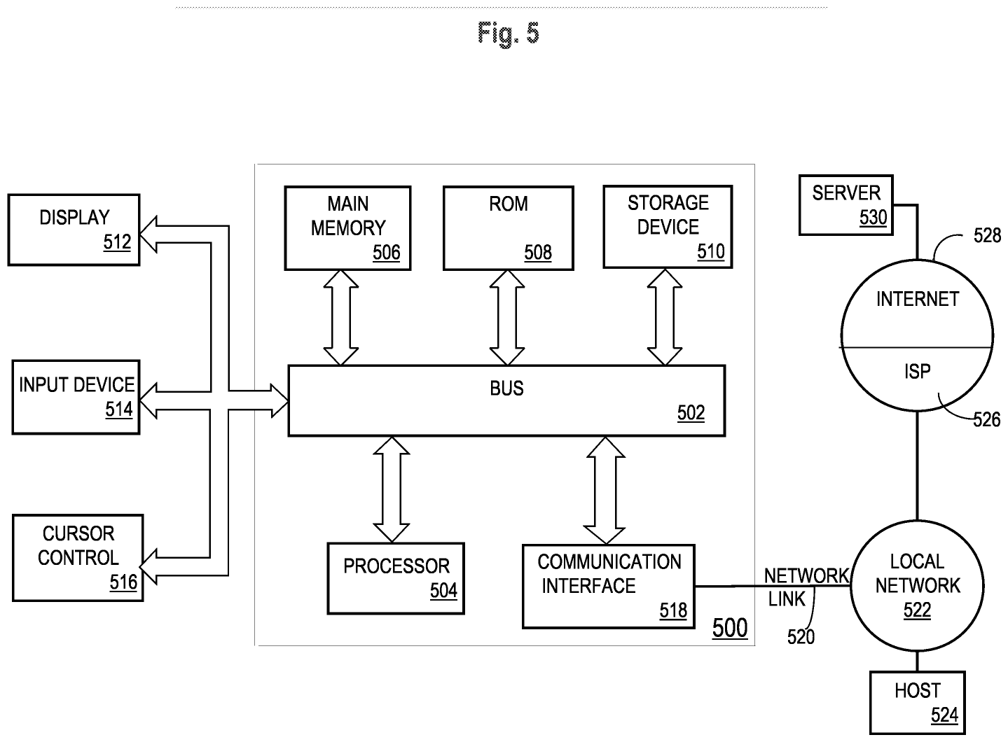

In one implementation, computing device 10 may be implemented in the manner shown in FIG. 5 and further described in other sections herein. FIG. 1A comprises one configuration of elements for the purpose of providing a clear example. In other configurations, more or less elements may be included. For example, the wireless relay device 4 may communicate with one or more server computers, such as one or more cloud servers, which store received data and communicate with client computing devices. As another example, the wearable elements may include wireless transmitters which send data directly to the computing device 10 without a wireless relay device 4.

In an implementation, server device 110 represents a server computer implemented to host services for executing machine learning models for predicting lameness for an animal. Specifically, the server device 110 hosts one or more machine learning models configured to receive, as input, observed sensor data from computing device 10 and determine whether the animal is experiencing a level of lameness based on the observed sensor data. Additional details describing the machine learning models and its related services are described in the MACHINE LEARNING MODEL section herein.

In an implementation, network 105 facilitates the exchange of communication and collaboration of data or any other type of information between computing device 10 and server device 110. Network 105 may be any type of network that provides communications, exchanges information, and/or facilitates the exchange of data between the computing device 10 and server device 110. For example, the network 105 may represent one or more local area networks (LANs), wide area networks (WANs), metropolitan area networks (MANs), global interconnected internetworks, such as the public internet, public switched telephone networks (“PSTN”), or any other suitable connections or combinations thereof that enable the computing device 10 to send and receive information to the server device 110. Each such network 105 uses or executes stored programs that implement internetworking protocols according to standards such as the Open Systems Interconnect (OSI) multi-layer networking model, including but not limited to Transmission Control Protocol (TCP) or User Datagram Protocol (UDP), Internet Protocol (IP), Hypertext Transfer Protocol (HTTP), and so forth. The network 105 may support a variety of electronic messaging formats, and may further support a variety of services and applications implemented on either computing device 10, server device 110, or both.

### Sensor Devices

FIG. 1B illustrates internal elements of an example sensor device of FIG. 1A. In an implementation, sensor devices 8 located on the paws of the animal 2 may comprise a body or housing that is configured to fit snugly over a foot or paw of the animal 2. In or integrated into the body or housing of sensor device 8 are a sensor 82, wireless transceiver 84, and power supply 86.

In an implementation, sensor devices 8 located on the paws of the animal 2 may represent shoes, boots or booties with sensors. The sensor devices 8 typically are configured to cause zero to minimal effect on the gait of the animal, or configured so that any effect on gait is the same for each limb. In various implementations, the sensor devices 8 may be disposable, or constructed of materials that are engineered to last approximately 1 week to 1 year in the typical clinical setting. Different implementations may comprise a plurality of same-sized sensor devices 8 that are intended for universal use with all animal limbs, or a plurality of different, specifically-sized sensor devices, or fully customized sensor devices that are fitted to a particular species, breed or individual animal.

In various implementations, sensor devices 8 may comprise sensors configured to be attached to a foot of an animal using different attachment methods. For example, miniaturized inertial motion units may be adhered to the nails of an animal using a glue, epoxy, or other adherence element. As another example a sensor device 8 may comprise sensors within a ring that is to be attached to a toe of an animal.

In other implementations sensor devices 8 may be attached to animal 2 using a strap or other hardware. For example, referring to FIG. 1A, sensor devices 8 located on a limb of animal 2 may be attached using a soft strap such that the sensor device 8 fits snugly on animal 2 in order to prevent slipping. Sensor devices 8 attached to animal 2 using straps may include sensor 82, wireless transceiver 84, and power supply 86. In another example, sensor device 8 may represent a sensor attached or within collar 6.

In various implementations, each sensor or array of sensors 82 may comprise any of force sensors, pressure sensors or hybrid sensors, inertial motion units, and/or other accelerometers in, on or associated with the sensor devices 8. As an example, force sensors may comprise sheets of material with contact leads for sensing a force and generating an analog electrical signal in response. The force sensors may be configured with a sheet or pressure plate between the foot of the animal and the sensor in order to measure the force placed on the ground by the foot. Additionally or alternatively, a force sensor may be configured with the sensor between the foot and the pressure plate, thereby measuring the ground reactive force on the limb.

In an implementation, each sensor or sensor array 82 is capable of measuring a total load or total ground reactive force per limb and can compartmentalize the paw strike. In an implementation, the application program 12 is programmed to collect data values for relative load or impulse of every limb on the same stride. In an implementation, each sensor 82 is capable of reporting a duration of a foot strike, or the computing device 10 or data relay device 4 is configured or programmed to calculate a duration of a foot strike based upon internal digital electronic clocks and data packets, messages or the content of data received from the sensors.

In an implementation, sensors 82 comprise dielectric electroactive polymers which provide output proportion to a magnitude of deformation of the polymer. Such sensors may be placed between two plates in order to measure the force with which a foot impacts the ground. In another implementation, sensors 82 comprises a plurality of pressure sensors arranged in an array, each of which being configured to determine if more than a threshold amount of pressure is imposed on the sensor. With a large array of pressure sensors, a system may compute the force of impact based on a percentage of sensors that activate during the impact.

In various implementations, the sensors 82 may comprise load cell strain gauges, piezoelectric load cells, resistive sensors or capacitance load cells. When load cell strain gauges are used, there may be four (4) strain gauges coupled using a Wheatstone bridge, or an array of load cells in each sensor device and coupled in parallel. In yet another implementation, the sensors 82 may comprise one or more viscous materials, which when loaded cause a change in one or more characteristics of the viscous materials such that the change in characteristics may be used as a sensor to signal pressure and/or load on the sensors 82.

In an implementation, each of the sensor devices 8 further comprises an accelerometer, which may be affixed or mounted at or near the hock or carpus. Additionally or alternatively, the accelerometer may be affixed closer to the heel of the foot or nail, which is expected to be the location of greatest change in acceleration of the foot or paw.

In an implementation, each of the sensor devices 8 further comprise one or more proximity sensors, which may be used in conjunction with accelerometers to determine proximity of each limb relative to one another, range of motion for each limb, distance travelled by each limb, and overall distance travelled by animal 2. The proximity sensors may be any type of proximity sensor including, but not limited to, inductive proximity sensors, capacitive proximity sensors, ultrasonic proximity sensors, and infrared proximity sensors. The proximity sensors may be used to measure the distance between, for example, the right front proximity sensor (right front paw) and the right rear proximity sensor (right rear paw) in order to detect changes in gait. If the right front proximity sensor and the right rear proximity sensor are detected to be closer to each other during activity, then this may be an indication of a shortened stride. Additionally, proximity sensors may be used to determine how spread the limbs in a pair. For example, if the distance between proximity sensors on the front legs widens, then the animal may be placing more load on each of the front legs. Similarly, if one back leg is loaded less, the contralateral leg will be placed more directly under the animal to better support the weight of animal 2. As a result, the affected limb will be placed further away from the animal's 2 center of mass/gravity to limit the load that limb supports.

In some implementations, the sensors 82 and/or accelerometer may comprise active electronic devices that are powered using disposable, replaceable or rechargeable batteries as the power supply 86 and that are mounted proximate to the sensors and electrically coupled to them. In some implementations, the sensor devices may comprise a switch that applies or disconnects power to the sensors; for example, magnetic reed switches may be used to facilitate non-contact switching through a sealed housing, or other external switches such as slide switches, toggles or pushbuttons. In some implementations, long-life sealed batteries such as lithium-ion batteries may be used and considered essentially disposable. Or, rechargeable batteries may be used with external capacitative charging, charging through a connector to a transformer or other external power supply, and/or charging through kinetic motion of the limb.

### Data Relay Device

In an implementation, the wireless data relay device 4 may comprise a compact housing that is capable of affixing to or integration with a collar 6, harness, lead, jacket, band, or other item that an animal may wear or carry. In one implementation, the wireless data relay device 4 comprises a central processing unit or microcontroller, wireless networking transceivers 42, 44, memory devices and programmed firmware that are collectively miniature and capable of integration into the collar 6 or other item. A dongle, capsule or other housing may be used and affixed to the item, or the electronic elements noted above may be in a housing that is sealed within a collar or other item that is capable of use with different animals.

The wireless networking transceivers 42, 44 may be programmed or configured to communicate with the sensor devices 8 and the computing device 10 through wireless network adapter 20. In an implementation, the transceivers communicate using different wireless protocols. For example, wireless networking transceiver 42 may communicate with sensor devices 8 through short distance wireless communication, such as Bluetooth, while wireless networking transceiver 44 communicates through a longer distance wireless communication protocol, such as WiFi, radio frequency, microwave, magnetic coupling, infrared transmission and/or any other means of wireless communication.

The wireless data relay device 4 may additionally include one or more proximity sensors, such as capacitive sensors, photoelectric sensors, inductive proximity sensors, and/or any of the other sensors described herein. Additionally and/or alternatively, the wireless data relay device 4 may comprise one or more sensor targets which can be sensed by one or more proximity sensors on other devices. For example a feeding and/or watering device may include a proximity sensor which is programmed or configured to detect wireless data relay device 4 when within the nominal range. Other implementations of data relay device 4 may include wired connections for attaching to a computing device. Thus, the data relay device 4 may receive data from the sensor devices and store the data until a wired connection is established between the data relay device 4 and a computing system.

While FIG. 1A depicts both sensor devices 8 and wireless data relay device 4, implementations may be implemented without wireless data relay device 4. For example, sensor devices 8 may comprise wireless networking transceivers and be programmed or configured to send data directly to a client computing device and/or cloud storage system.

### Application Program

In an implementation, the application program 12 is configured or programmed to receive and format sensor data, from the data relay device 4, and provide the sensor data to the server device 110 for determining a level of lameness for the animal 2. For example, the application program 12 may receive raw data, from the data relay device 4, indicating foot strikes and force of each foot strike. The application program 12 may process the raw data to generate processed data that includes force data for the amount of force applied by each paw, durations of foot strikes based on the start and stop time of a foot strike, durations between foot strikes, as well as correlating GPS data to determine the amount of movement associated with observed gait of animal 2. In another example, the application program 12 may receive accelerometer information from various sensor devices 8 enabled to capture kinematic data. The application program 12 may associate the received kinematic data with captured video to generate sets of sensor data that may be provided to the server device 110.

The application program 12 may receive from the server device 110, lameness values indicating the severity of the lameness detected as well as one or more recommendations to treat the lameness detected. In an implementation, the application program 12 is configured to display information about the lameness detected. For example, the application program 12 may display the detected lameness in the form of relative paw strike force or impulse in relation to the contralateral and other paws. In another example, the application program 12 may display laterality weight bearing/impulse/other kinematic or kinetic gait data percentage values, fore/rear limb weight bearing percentage values, and individual weight bearing percentage values.

In another implementation, prior to receiving lameness values from the server device 110, the application program 12 may provide a real-time visualization of the data that the sensors devices 8 collect. For example, the application program 12 may display, in real-time, a percentage of weight that is bearing on each limb.

In an implementation, the application program 12 is further configured or programmed to display a set of treatment recommendations received from the server device 110, including, but not limited to a set of potential or current injuries that may be causing lameness, sets of rehabilitative exercises that may help improve lameness, and a set of activities to avoid as these activities may exacerbate lameness.

In an implementation, the application program 12 is configured or programmed to support creating and storing tests or trials of specific animals that are named or identified, including receiving start and stop signals via input to the computing device, and to compute and generate statistical data such as average values during the trial and/or trend values during the trial, with respect to any of the data values that are identified above.

In an implementation, the application program 12 is configured or programmed to obtain the geo-location data from the GPS transceiver 18 and to calculate velocity values that represent a velocity of animal 2 over time. Velocity values may be generated and stored in association with weight bearing values and/or other kinetic/kinematic values that are identified from the sensors 82 of the sensor devices 8. The application program 12 may calculate and display a change in weight bearing at different speeds of the animal 2, with or without a representation of a time axis.

In an implementation, the application program 12 is configured to access camera 16 that is integrated in or coupled to the computing device 10 and to cause initiating a video recording at the same time or in association with starting testing or a trial. For example, when the computing device 10 is a mobile computing device such as a smartphone or tablet computer having an integrated camera 16, the application program 12 is configured or programmed to programmatically call or signal a service, function or primitive of the operating system 14 of the computing device to access the camera and/or trigger a camera recording function. Such a call or signal may be generated when a test or trial starts, or asynchronously with respect to the initiation of data gathering. The particular way of triggering a video recording is not critical.

In an implementation, the application program 12 is configured or programmed to obtain a timestamp value, based on a system clock of the computing device 10, at the time that the video recording starts and to store the timestamp value in association with a dataset representing a continuous set of data from sensors 82 that is collected during a test, trial or other period that coincides with the recording. In this manner, the application program 12 is configured or programmed to associate a start of the video recording with a particular set of first data values or starting data values that correspond to the start of the video recording.

Consequently, the application program 12 also may be configured or programmed to replay the video recording on a display device of the computing device 10, in synchronization with continuous display of a changing graph of the data that was gathered at the same time as the recording occurred. In an implementation, the application program 12 is configured or programmed to detect when the sensor data indicates asymmetry, thereby indirectly indicating asymmetry in weight bearing on different limbs of the animal 2, and to generate and display a marker, alert, notification or other signal in a portion of the computer display. The marker, alert, notification or other signal may be displayed intermittently as asymmetry is detected or not detected, and may be visually or graphically superimposed over the replayed video recording of the animal 2.

### Server Device

FIG. 1C depicts an example of a server device implemented to execute one or more machine learning models for predicting gait asymmetry of an animal and for predicting lameness severity and treatment recommendations based on observed sensor data from one or more sensor devices associated with the animal, according to an implementation. Server device 110 is shown as communicatively coupled to network 105 and includes a machine learning management service 112, an asymmetry prediction model 114, a lameness recommendation model 116, and a data repository 118. In other implementations, server device 110 may be implemented within a server cloud of computing resources. Cloud computing is described in the CLOUD COMPUTING section herein.

In an implementation, the machine learning management service 112 represents a computer process or program configured to implement an asymmetry prediction model 114 and a lameness recommendation model 116. The asymmetry prediction model 114 represents a machine learning model configured to identify gait asymmetry affecting different limbs of an animal. The lameness recommendation model 116 is a machine learning model implemented to predict lameness severity for as animal and generate treatment recommendations for the animal.

The machine learning management service 112 may be implemented in any number of ways, including as a stand-alone application running on server device 110, web services running on server device 110, etc. Embodiments of the machine learning management service 112 described herein, may be comprised of a combination of software, and allocated of resources from the server device 110. Specifically, an application is a combination of integrated software components and an allocation of computational resources, such as memory, and/or processes on the computing device for executing the integrated software components on a processor, the combination of the software and computational resources being dedicated to performing the stated functions of the application.

In an implementation, data repository 118 represents a storage medium configured to store observed sensor data collected by the application program 12 and characteristics of animal 2. Additionally, data repository 118 may store any medical information for animal 2, including, but not limited to, medical history from veterinarians, historical sensor data previously collected, any observed information collected by an owner of animal 2, and any other information relevant to health conditions of animal 2. The data repository 118 may comprise a database. As used herein, the term “database” may refer to either a body of data, a relational database management system (RDBMS), or to both. As used herein, a database may comprise any collection of data including hierarchical databases, relational databases, flat file databases, object-relational databases, object-oriented databases, distributed databases, and any other structured collection of records or data that is stored in a computer system. Examples of RDBMS's include, but are not limited to including, ORACLE®, MYSQL, IBM® DB2, MICROSOFT® SQL SERVER, SYBASE®, and POSTGRESQL databases. However, any database may be used that enables the systems and methods described herein.

### Machine Learning Model—Asymmetry Prediction Model

FIG. 1C depicts two machine learning models, the asymmetry prediction model 114 and the lameness recommendation model 116. In an implementation, the asymmetry prediction model 114 is implemented to receive, as input, observed sensor data from the application program 12. The observed sensor data may include raw data from one or more sensor devices 8 for animal 2 and processed data processed by the application program 12. Examples of the observed sensor data may include, but are not limited to, force data representing amounts of force applied from each paw, kinematic data collected from one or more accelerometers, durations of foot strikes, durations between foot strikes, captured video data of movement of animal 2 during activity, GPS data describing distances travelled, distances between paw strikes, distances between limb sensors as animal 2 moves, amplitude measured from paw strikes, frequency of paw strikes, and any other observed data. Additionally, input for the asymmetry prediction model 114 may include characteristics of animal 2, including, but not limited to, the age, weight, breed, medical history, activity history, and any other relevant characteristics of animal 2.

The asymmetry prediction model 114 may use the characteristics of animal 2 to classify the animal's observed sensor data in order to evaluate the severity of asymmetry found from the set of observed sensor data. The asymmetry prediction model 114 may implement different lameness thresholds for different breeds, sizes, conformations, and ages of animals. Additionally, prior medical history may be factored when evaluating observed sensor data to determine whether animal 2 is experiencing lameness. For example, the asymmetry prediction model 114 may use different lameness thresholds for a healthy young Labrador, than an older Labrador that has been experiencing arthritis in its hip.

In an implementation, the asymmetry prediction model 114 may analyze the observed sensor data and determine whether the animal 2 is experiencing lameness based on the characteristics of animal 2. The asymmetry prediction model 114 may be trained to analyze force, pressure, impulse, or other kinematic or kinetic values to determine whether a limb is affected by lameness. For instance, the asymmetry prediction model 114 may compare average force exhibited by contralateral limbs over a period of time to determine whether gait asymmetry exists. In another example, kinematic and force data may be used to identify each gait as a function of a pattern of time each limb spends on the ground versus aloft compared with the contralateral and other limbs. For example, a trot may be defined as occurring when diagonally opposite limbs are moving in unison. Thus, the front left leg and rear left leg are both aloft and on the ground at the same time. A gallop may be defined as occurring when contralateral limbs are lifted/placed on the ground in succession, such that time spent aloft and on the ground for contralateral limbs overlap but do not exactly match. A run may be defined as occurring when the time limbs spend aloft exceeds the time the limbs spend on the ground. The asymmetry prediction model 114 may identify changes in patterns of limb movement to determine changes in gait. For example, the asymmetry prediction model 114 may identify a first pattern of limb movement over a first period of time. The asymmetry prediction model 114 may determine points in time where the limb movement does not match the prior pattern.

In an implementation, the asymmetry prediction model 114 uses movement data, position data, and/or distance values derived from multiple sensor data to determine distance traveled by individual limbs and/or by the animal. Additionally, the asymmetry prediction model 114 may compute an average velocity for each limb as the quotient of the distance traveled and the time during which the distance was traveled. The asymmetry prediction model 114 may use computed velocities to determine whether a limb is affected by lameness. For example, if the difference in average velocities and/or maximum velocities of contralateral limbs is greater than an expected threshold value for the same animal at a different time or similar animals, the asymmetry prediction model 114 may determine that the limb with a different velocity is being affected by lameness.

Based on the observed sensor data from the application program 12, the asymmetry prediction model 114 determines levels of gait asymmetry for animal 2. In some implementations, the input sensor data may include only certain types of data, such as kinematic data or force data. The asymmetry prediction model 114 is trained to generate asymmetry predictions based on a variety of data regardless of if each type of data is provided as input. For example, asymmetry prediction model 114 is trained to provide asymmetry predictions solely on kinematic data. In another example the asymmetry prediction model 114 is trained to provide asymmetry predictions solely on force data.

In an implementation, the asymmetry prediction model 114 may determine whether animal 2 is exhibiting lameness based on the different types of gait observed. For example, the asymmetry prediction model 114 may determine that animal 2 exhibits minimal lameness during a walk but experience moderate lameness during a trot. Additionally, the asymmetry prediction model 114 may be further trained to determine when during a specific type of gait lameness occurs. For example, animal 2 may not exhibit any lameness during the first five minutes of a trot but after five minutes, animal 2 begins to exhibit lameness. The asymmetry prediction model 114 may be implemented to output different levels of gait asymmetry for animal 2 based on the type of activity.

In an implementation, the asymmetry prediction model 114 may implement any variety of prediction models, including, but not limited to, binary classification model, a logistic regression model, a linear regression model, an artificial neural network, decision trees, support vector machines (SVM), Bayesian networks, and any other type of machine learning model. Training the asymmetry prediction model is described in the TRAINING MACHINE LEARNING MODELS section herein.

### Machine Learning Model—Lameness Recommendation Model

In an implementation, the lameness recommendation model 116 is implemented to receive, as input, the asymmetry prediction values for each of the limbs of animal 2 from the asymmetry prediction model 114. The lameness recommendation model 116 uses the asymmetry prediction values and characteristics of animal 2 to generate a set of lameness recommendations that predict the severity of the lameness and treatment options for animal 2. For example, the lameness recommendation model 116 analyzes the asymmetry prediction values in conjunction with the characteristics of animal 2 and determines a set of recommendations for treatment. Output recommendations from the lameness recommendation model 116 may be based on the severity of the asymmetry, breed of the animal, weight and age of the animal, prior medical history of the animal, and any other animal characteristics that may be relevant to predicting the effect of the asymmetry on the animal's long-term health.

In an implementation, output from the lameness recommendation model 116 may include a set of potential injuries that may be caused by the identified lameness and the likelihood of each potential injury occurring. For example, if the lameness recommendation model 116 determines that animal 2 may be susceptible to a cranial cruciate ligament (CCL) tear on its rear right limb, the output may include a percentage chance for the CCL tear as well as a prediction on when the injury may occur. For instance, the output may state that animal 2 is 65% susceptible to an CCL tear within the next six months. Output from the lameness recommendation model 116 may be sorted in terms of level of severity where potential injuries that are more likely to occur are presented first or are marked as high priority.

In another implementation, the lameness recommendation model 116 may output a set of recommended exercises for animal 2 based on the level of lameness detected. For example, if animal 2 is experiencing lameness during recovery of an injury, the lameness recommendation model 116 may determine, based on the lameness identified and the medical history of animal 2, that a set of rehabilitation exercises should be performed to help animal 2 recover from their existing injury and reduce the identified lameness.

In another implementation, the lameness recommendation model 116 may output a set of prohibited activities, where the set of prohibited activities are activities that may worsen the identified lameness. For example, if sensor data indicates that animal 2 experiences increased lameness after swimming then the lameness recommendation model 116 may recommend avoiding swimming exercises for animal 2.

In an implementation, the lameness recommendation model 116 may receive, as input, the asymmetry prediction values for each of the limbs of animal 2, where the asymmetry prediction values include the type of gait observed. For example, a first set of asymmetry prediction values may be associated with walking, while a second set of asymmetry prediction values may be associated with trotting. The lameness recommendation model 116 may then determine, based on the asymmetry prediction values and their associated types of gait, that animal 2 is spending more time trotting than walking, when compared to previous observations. This may be indicative of animal 2 avoiding a walk due to pain or other mobility restrictions. The lameness recommendation model 116 may be configured or programmed to provide evaluation recommendations to suggest that the owner or trainer have animal 2 walk more in order to observe any potential lameness that may be causing the changes in activity of animal 2. If, for example, the owner or trainer causes animal 2 to walk more, then the one or more sensor devices 8 may observe instances where animal 2 exhibits lameness during the walk. The observations from the one or more sensor devices 8 may be evaluated by the asymmetry prediction model 114 to determine whether or not animal 2 is exhibiting lameness during walking.

### Training Machine Learning Models

In an implementation, the machine learning management service 112 is implemented to train the machine learning models using a corpus of collected sensor data for a corpus of animals and animal breeds. For example, the asymmetry prediction model 114 may be trained using a corpus of collected sensor data that includes a variety of sensor data that includes, but is not limited to, force and pressure data, kinematic data, video data, audio data, and any another data that may be observed using a variety of sensors in a variety of environmental conditions. The corpus of animals and animal breeds may include collecting sensor data from a variety of dogs, cats, horses, and other animals. Additionally, the corpus of animals may include animals of a variety of sizes, weights, ages, breeds, as well as animals that have experienced a variety of medical conditions. The sensor data may be labelled with a level of gait asymmetry for animal 2.

In an implementation, the lameness recommendation model 116 may by trained using a corpus of gait asymmetry data for a corpus of animals and animal breeds where the corpus of gait asymmetry data is labelled with underlying medical conditions. For example, the training data may include a set of gait asymmetry values for a Labrador, where the gait asymmetry values are labelled with specific injuries that may occur and the probability for those injuries occurring based on the gait asymmetry values and specific to the Labrador and its specific medical characteristics.

### Process of Use

In an implementation, the system described herein is used as part of a diagnostic process that is capable of reporting an assessment of lameness and providing a set of treatment recommendations. In one approach, the sensor devices are fitted to the animal, optionally including applying or turning on power to the sensors or sensor devices. Before or afterward, the wireless data relay device, if used, is affixed to the animal, such as by attaching a collar or other wearable item. The application program is launched on a compatible computing device, and establishes wireless communication with the sensors and optionally with the wireless data relay device; in this manner, the application program begins requesting, obtaining or receiving a stream of data values either directly from the sensors or indirectly via the data relay device. Data may comprise raw sensor values indicating kinematic movement, pressure or force, or transformed data indicating weight, together with unique identifiers of which limb, paw or sensor reported the data.

In an implementation, the system is initially calibrated using one or more external devices. For example, the animal may be fitted with the sensor devices initially. The sensors for the sensor devices may be initially zeroed out with the animal lying down or in a position where little to no force is placed on the sensors in the sensor devices. The animal may then be placed on a digital scale to obtain weight measurements while a stream of data values is obtained from the sensor devices. The stream of data values representing force from each of the sensor devices may be aggregated and compared to weight data from the digital scale. The system may then be calibrated such that the total forces from the sensor devices aggregate to the measured weight of the animal.

Weight values may be manually input from reading of the scale and/or automatically sent from the scale to an external device. For example, the scale may include a proximity sensor which is configured to detect proximity of the wireless relay device 4. The scale may identify an account of wireless relay device 4 based on data transmitted from wireless relay device 4 and send weight data to a computing device associated with the account and/or to a server system such that the data can be associated with the account. Additionally or alternatively, a tag worn by the animal may comprise a scannable code, such as a bar code or QR code which can be scanned by a scanner on the scale to associate weight data with the animal.

In a typical usage scenario, the animal may be caused to walk a short distance over any available surface and in any available location. A pad, plate or other specialized surface is not required. As an example, an owner, caretaker or animal healthcare provider may lead or call the animal to cause walking. During gait of the animal, the sensors continuously produce output data indicating pressure, force, and/or other kinematic data or kinetic data such as acceleration or max velocity or provide data in response to signals, polls or calls by the data relay device or the computing device. In an implementation, the application program is configured or programmed to display a graph, graphical user interface, or other visual output. In various implementations, the visual output may comprise raw sensor values indicating pressure or force, or transformed data indicating weight or impulse, together with unique identifiers of which limb, paw or sensor reported the data. Or, the visual output may comprise graphs, charts or tables (graphical representation of current gait overlaid on breed/conformation-specific representations or historical ones) specifying either raw data or transformed data, alone or in conjunction with a video player window that shows a video recording of the animal as the animal is moving.

In some implementations, the application program 12 is configured or programmed to display calculated gait asymmetry values provided by the machine learning management service 112 and one or more treatment recommendations in the visual display of the computing device 10. The treatment recommendations may comprise, for example, a list of possible diagnoses that are associated with the particular species of animal 2, the data values that were collected, and treatment options for animal 2. Treatment options may include a visit to a veterinarian, specific exercise to rehabilitate any identified injury, and potential activities to avoid based on identified injuries. For example, the application program 12 may display information including, one or more of the maximum velocity and/or other measured values, velocity versus time, number of activated sensors of a sensor array, length of time in various gaits, changes in symmetry during gaits, gait changes, changes in gait characteristics over time, possibilities of bilateral disease based on changes in limb values, diseases or pathologies affecting forelimb versus hind limb, compensatory changes in kinetic values, and/or a degree of certainty of diagnosis based on the received values.

In some implementations, the application program 12 is configured or programmed to gather sensor output data and geo-location data over an extended period of time in order to determine regular patterns of animal 2. For example, a healthy animal 2, at a specific park/trail, may spend 20% of its time at a walk pace, 75% of its time at a trot pace, and 5% of its time doing other things. The application program 12 may determine a baseline level of activity for animal 2 based on the observations from the specific park/trail. Then application program 12 may use the determined baseline level of activity to identify if and when animal 2 deviates from its baseline. For instance, a year later, animal 2 may be observed at the specific park/trail as spending 45% of its time at a walk pace. The application program 12 may be configured or programmed to generate a notification when animal 2's activity deviates from the determined baseline level and type of activity for a specific geo-location. Implementations of detecting deviations in baseline level and type of activity are not limited to a decrease in activity. In some instances, the application program 12 may detect that animal 2 is trotting more than running, which could also be indicative of subtle changes and potential lameness. Any changes in baseline level and type of activity may trigger the need for additional observations and tests for animal 2.

### Process Overview

The systems described herein may be used to detect lameness, in terms of gait asymmetry, during one or more movement events. Movement events may refer to discrete instances of movement or non-movement for an animal. For example, movement events may be separated by instances of the animal standing, sitting, lying down, or a change to a different type of movement event. Additionally and/or alternatively, movement events may refer to discrete instances of movement at a particular gait. Thus, if an animal is initially moving in an asymmetrical walking gait and shifts to a running trot, the time spent in the walking gait would be considered a first movement event while the time spent in the running trot would be considered a second movement event.

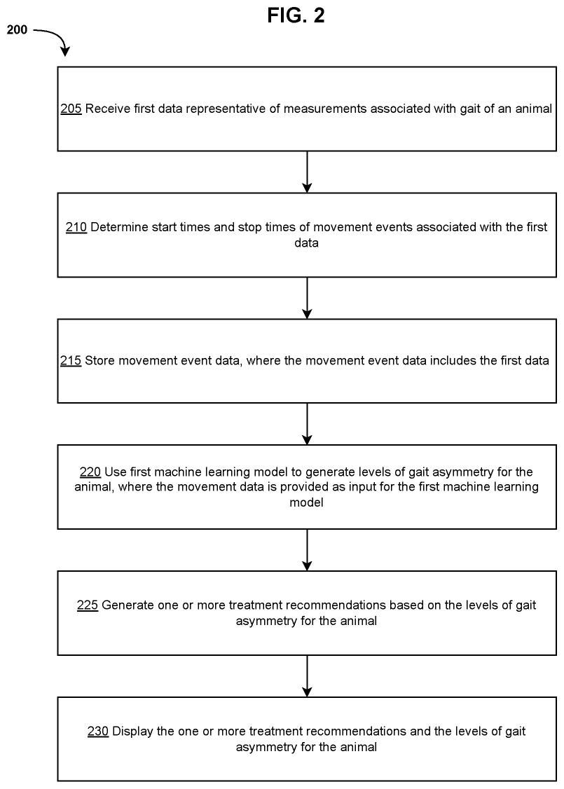

FIG. 2 depicts a flowchart for automatically detecting lameness in an animal based on movement events and generating a recommended treatment and management options, according to an implementation. The steps of the process as shown in FIG. 2 may be implemented using processor-executable instructions that are stored in computer memory. For the purposes of providing a clear example, the steps of FIG. 2 are described as being performed by services executing on either the computing device 10 and/or server device 110. For the purposes of clarity, process 200 described may be performed with more or fewer steps than described in FIG. 2.

In an implementation, sensor devices 8 on animal 2 detect horizontal movement. For example, the sensor devices 8 may include an accelerometer, gyroscope, and/or inertial measurement unit which sends acceleration data to the wireless data relay device 4. The wireless data relay device 4 may comprise digitally programmed logic which is configured to determine whether the acceleration data indicates horizontal movement. For example, acceleration in a direction perpendicular to the constant downward acceleration as measured by the accelerometers and/or other gravitometers may be identified as horizontal acceleration and used to compute horizontal velocity of the animal. Additionally and/or alternatively, the wireless data relay device may send acceleration data to an external computing device which is programmed or configured to determine whether the acceleration data indicates horizontal movement. Thus, as used herein, determinations made by the system may be performed at the wireless data relay 4 and/or at an external computing device.

At step 205, process 200 receives first data representative of measurements associated with gait of the animal. In an implementation, the data relay device 4, receives from the sensor devices 8 observed measurements associated with movement of animal 2. For example, the data relay device 4 may continuously receive measurements of acceleration, vertical impact, vertical impulse, pressure, position, and or other motion related values. The measurements may be received as time series data which include temporal data in conjunction with the measured accelerations, vertical impulses, pressure, and/or position. The measurements may be continuously sent from the sensor devices 8 prior to the movement event and/or sent from the sensor devices 8 in response to a detection of a movement event. In an implementation, the data relay device 4 sends the first data to the application program 12, running on computing device 10. The application program 10 may determine a gait of the animal. For example, the application program 10 may identify, from the first data, measurements corresponding to each limb. Based on the measurements, the application program 10 determines motion of each limb. Based on motion and placement of each limb in relation to the other limbs, the application program 10 may determine the gait and/or movement type of the animal.

In another implementation, the data relay device 4, may receive second data from another set of sensor devices 8 which contain additional observed measurements associated with movement of animal 2. For example, the first data may include kinematic based measurements, while the second data includes force-based measurements. In another example, the first data may correspond to measurements from a first limb, while the second data may correspond to measurements from a second limb. In yet other implementations, the data relay device 4 may receive a plurality of data from a plurality of sensor devices 8 located on various spots on animal 2.

At step 210, process 200 determines a start and stop time of a movement event. In an implementation, the application program 10 determines the start and stop time of a movement event. The start and stop times may be determined based on input from an external computing device, based on initiation/cessation of horizontal motion, and/or beginning/ending of movement at a particular gait. For example, if a running gait is defined as a movement where the limbs spend a greater amount of time off the ground than on the ground, then the beginning of the movement event for a running gait may be determined to be a point in time where the amount of time each limb spends off the ground exceeds the amount of time the limb spent on the ground prior.

At step 215, process 200 stores measurements for movement events. In an implementation, the application program 12 stores measurements for movement events in data repository 118. For example, the application program 12 may store acceleration, vertical impulse, and/or pressure/force measurements for each of the limbs between the identified start time of the movement event and the identified end time of the movement event. The application program 12 may additionally compute one or more values from the measurement data. For example, using the acceleration and temporal data, the application program 12 may compute velocities with respect to time and/or positions with respect to time. Additionally, and/or alternatively, the system may compute averages, maximums, and/or minimums of measured values, such as acceleration, vertical impact/impulse, and/or pressure. In another implementation, the application program 12 stores the measurements for movement events within local storage drives within computing device 10.

At step 220, process 200 uses a first machine learning model to generate levels of gait asymmetry for the animal, where the movement data is provided as input for the first machine learning model. In an implementation, the machine learning management service 112 initiates a request to the asymmetry prediction model 114 to generate levels of gait asymmetry for the animal. The machine learning management service 112 provides the movement data as input to machine learning management service 112. Additionally, the machine learning management service 112 may provide characteristics of animal 2 and medical history of animal 2 to the asymmetry prediction model 114. In an implementation, the asymmetry prediction model 114 generates output that contains one or more predicted levels of gait asymmetry. The levels of gait asymmetry may be used to determine whether the animal 2 is experiencing lameness that may otherwise not be detected from a visual analysis of the animal 2.

At step 225, process 200 generates one or more treatment recommendations based on the levels of gait asymmetry. In an implementation, the machine learning management service 112 may make a request to the lameness recommendation model 116 to generate one or more treatment recommendations. The machine learning management service 112 may provide, as input for the lameness recommendation model 116, the levels of gait asymmetry calculated by the asymmetry prediction model 114 and characteristics of animal 2. The characteristics of animal 2 may include the animal's 2 species, breed, age, weight, medical history, and any other information that may be used to identify potential treatment options.

In an implementation, the lameness recommendation model 116 uses the asymmetry prediction values and characteristics of animal 2 to generate a set of lameness recommendations that predict the severity of the lameness and treatment options for animal 2. The output from the lameness recommendation model 116 may include a set of potential injuries that may be caused by the identified lameness and the likelihood of each potential injury occurring. The output may also include a set of recommended exercises for animal 2 based on the level of lameness detected. Additionally, the output may include a set of prohibited activities, where the set of prohibited activities are activities that may worsen the identified lameness.

At step 230, process 200 displays the one or more treatment recommendations and the levels of gait asymmetry for the animal. In an implementation, the application program 12 may cause display within a graphical user interface of a client computing device, the one or more treatment recommendations and the levels of gait asymmetry for the animal.

In another implementation, the application program 12 may also cause display, within a graphical user interface, measurement values for contralateral limbs. For example, the application program 12 may display any of acceleration values, force values, vertical impulse values, distance v. time graphs, symmetry v. time graphs, and/or acceleration v. time graphs on a client computing device. Methods for displaying measurement values and/or computed values on a client computing device are described further herein.

### Display Examples

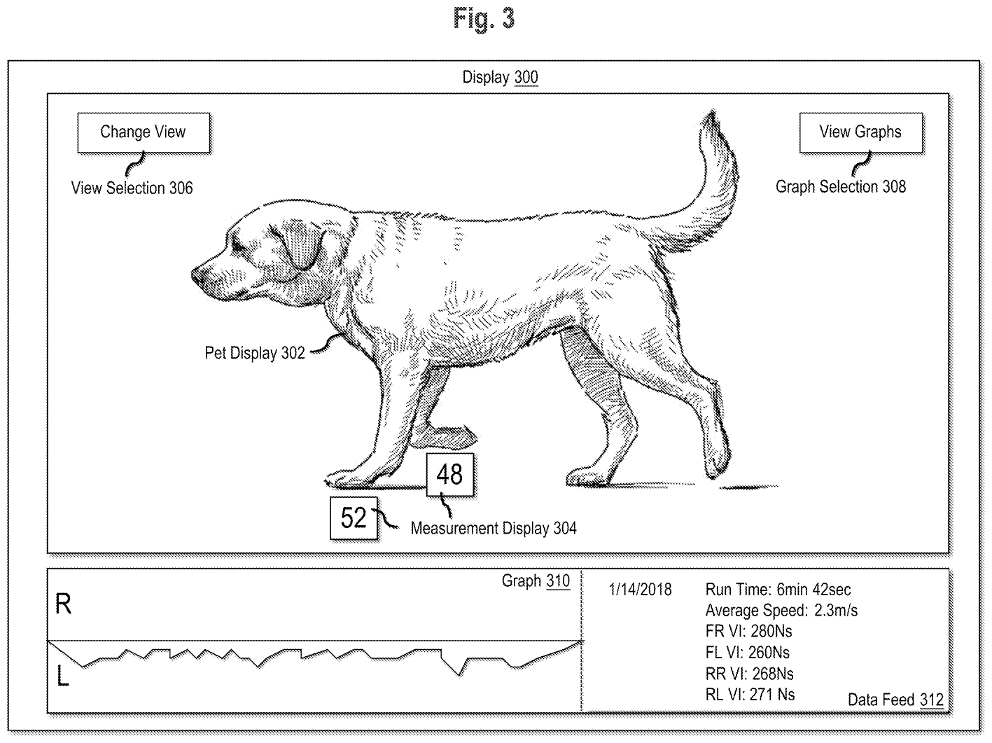

In an implementation, the system generates a display on a client computing device which is used to view captured and/or computed data regarding the animal. FIG. 3 illustrates an example graphical user interface (GUI) with example visualization of lameness data. Display 300 comprises pet display 302, measurement display 304, view selection 306, graph selection 308, graph 310, and data feed 312.

Pet display 302 comprises an image of the animal which is being monitored. Pet display may include a generic outline of an animal. For example, if the monitored animal is a dog, the system may display a generic outline of a dog. In an implementation, the system uses images of the animal being monitored. For example, an application running on a client computing device may initially request images of the monitored animal from one or more angles while the pet is standing, walking, and/or otherwise upright so that one or more limbs are visible. The application may utilize a camera on the client computing device, such as an internal camera of a smart phone, and/or request upload of an image. When the application receives a requested image, the application may request identification of individual limbs, such as through use of a cursor and/or touch screen. The application may then store the image of the animal with metadata describing the view of the animal, such as front view or side view, and locations of the one or more identified limbs.

Measurement display 304 comprises a display of one or more measurements and/or computed values for individual limbs. Values displayed in measurement display 304 may include absolute and/or relative vertical impulse, acceleration, velocity, and/or time limb spends on the ground/aloft. The system may be programmed or configured to display one type of value and/or to switch between value types based. For example, the interface may include an option to toggle between displaying vertical impulse, acceleration, velocity, and or time limb spends on the ground/aloft. Various displays may show different limbs. For example, one display may show only the front limbs while a different display shows only rear limbs.

The measurements of measurement display 304 may be displayed over the individual limb to which the measurement refers. For example, a value displayed over the right front limb may be a measurement and/or computed value for the right front limb. In the example of FIG. 3, the system displays values representing a percentage of vertical impact with respect to total animal weight on each limb. Thus, the front right limb was measured to produce 48% of the vertical impact of the dog's weight while the front left limb was measured to produce 52% of the vertical impact of the dog's weight.

In an implementation, the display may additionally identify the gait of the animal during the movement event. For example, the gait of the animal may be displayed above an image of the animal or superimposed on an image of the animal. In implementations where the interface displays data regarding the pet in real time, the display may update to show the new gait of the animal when the device determines that the gait of the animal has changed.

The gait of the animal may be used to determine when to begin capturing data to be relayed to the computing device. For example, the system may determine whether a steady gait has been reached, such as by identifying a repetition of a gait over a particular number of strides. Additionally or alternatively, a calibration step may be performed where a specific gait for an animal is recorded during movement of the animal. The system may identify the specific gait as a “usual” gait for the animal and begin recording capturing data to be relayed to the client computing device when the animal reaches the “usual” gait.

View selection 306 may be displayed on the graphical user interface for changing the view of the animal through the graphical user interface. For example, the system may include multiple views of the pet, including a front view, a side view, and a back view. The system may switch between the views for comparisons of different limbs. Additionally, the system may switch between different types of views.

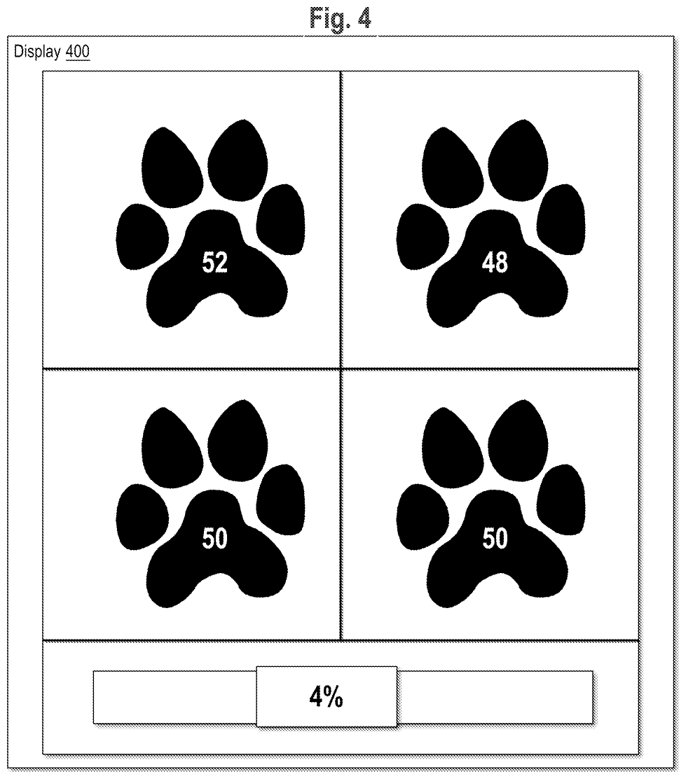

For example, FIG. 4 depicts a graphical user interface depicting comparisons of data for the limbs of an animal. The display of FIG. 4 may be selected as a different view and/or may act as an alternative view for the system. Display 400 includes depictions of paws for each limb and a value for each paw indicating one of the measurements and/or computations for the corresponding limb. The view of FIG. 4 allows for easy comparison of the individual limbs. Additionally, the view may include a slider which visually indicates asymmetry of the limbs. For example, the slider at the bottom of the display of FIG. 4 is displayed as being balanced slightly to the left to indicate that more pressure is being placed on the left front limb than the right front limb. Additionally, the slider includes a value indicating the percentage difference between the value for the left front leg and the right front leg.

In an implementation, a display similar to FIG. 4 may be used to show a center of gravity based on load bearing. The center of gravity may refer to the two-dimensional center of downward force or pressure. For example, the system may compute a location of the center of gravity of the animal based on the load placed on each foot. Thus, if 60% of an animal's load is placed on the front limbs, 40% on the rear limbs, and no asymmetry exists between contralateral limbs, the icon may be placed slightly higher than the center point between the four displayed paw prints. A video display may additionally depict the change in the center of gravity over time by depicting movement of the icon from indicating a center of gravity initially to indicating the recent measurement of center of gravity.

Returning to FIG. 3, graph selection 308 comprises an option for viewing different graphs. The graphs may include line plots depicting changes in values over time with respect to one or more limbs. For example, a line plot may include a line for the front left limb and a line for the front right limb. Additionally and/or alternatively, a graph may include a single line denoting differences in symmetry for different values. Thus, when the values are the same for contralateral limbs, the line may be centered in the graph. When the value for a first limb begins exceeding the value for its contralateral limb, the line in the graph may begin moving towards the side representing the first limb.

Differences in symmetry may additionally be captured for different gaits and displayed through the computing device. For example, the system may determine an average symmetry difference value for each gait over a period of time, such as a week. The system may display the different symmetry values, thereby indicating which gaits have the higher impact on the symmetry of the animal.

Acceleration values collected over different periods of time may be used to generate graphs of maximum acceleration versus time for each limb. A graph depicting changes in maximum acceleration over time allows the system to visually indicate to a user the changes in the animal's movement over time and to indicate times where the variance between limbs was particularly pronounced. Vertical impact versus time graphs may additionally be used to display changes to the animal's movement with respect to time. Graphs may be displayed individually and/or overlaid on each other. For example, a display may show side by side superimposed graphs of vertical impulse, maximum velocity, and acceleration versus time.

A distance versus time graph may visually indicate when an animal is moving one limb slower than the other, as the graph would visually indicate that one of the limbs took a longer amount of time to cover a same distance as a contralateral limb. The distance versus time graph thus visually indicates that an animal has been moving one limb faster than another at different points in time. Additionally, the distances versus time graph may be used to provide velocities of the individual legs which can be compared to the velocity of the animal.

The system may also display a symmetry versus time graph which visually depicts differences between the limbs over time. For example, a symmetry graph for maximum acceleration would show differences in the accelerations of contralateral limbs over time. Thus, if at a time A, both limbs had the same acceleration, the graph would show neutral symmetry for time A. If at time B, the system computed a 5% difference in acceleration between the limbs, the graph would depict a 5% difference at time B.

In an implementation, the system visually identifies portions of a graph that indicate lameness in the animal. For example, the system may highlight a portion of an acceleration graph where the differences in maximum acceleration between two contralateral limbs exceeded a stored threshold value. By visually altering the graph based on a determination of lameness, the system generates a dynamic graphical user interface which is effective in not only identifying lameness, but also displaying evidence of lameness.

Graph 310 comprises a graph that is displayed with interface 300. Graph 310 may include any of the graphs described above. In FIG. 3, graph 310 is a graph depicting relative values over time. Thus, as the animal begins favoring a particular leg, the line of the graph begins moving away from center. The graph of 310 may be a fixed graph type and/or a changeable graph. Thus, a user may select a different graph type to be displayed in display 300.

Graph 310 and measurement display 304 may be based on the totality of data gathered, specific instances of data gathered, and/or most recent instances of data gathered. For example, measurement display 304 may depict overall averages of vertical impact of the limbs while the graph 310 displays changes of vertical impact on the limbs over time. As another example, the system may display measurement data 304 and graph 310 based on a current and/or most recent movement event. For a current movement event, the system may continuously update graph 310 and measurement 304 based on newly measured values.

In an implementation, the graph comprises a visual representation of differences between current values and past values. For example, an overlaid bar graph may depict pressures placed on each limb currently in one color and average pressures placed on each limb in the past in a different color, thereby depicting a visual indication of the changes of the animal over time.

In an implementation, the system displays options for selecting different time periods, gaits, and/or tracked values for graph 310 and/or measurement display 304. For example, in response to a selection of a “trot” gait, the system may display average values obtained during a trot gait in measurement display 304 and changes in the values during the trot gait in graph 310. As another example, the system may respond to a selection of a particular movement event and/or a selection of a particular time period by displaying data values for the movement event and/or particular time period.

Data feed 312 comprises a display of data values. Data feed 312 may include data values for movement events, periods of times, and/or different gaits. The data feed 312 may include an identification of a time period, movement event, and/or gait for each set of values. For example, in FIG. 3, data feed 312 comprises data values for a run performed on Jan. 14, 2018. The data values include the run time for the pet, the average speed of the pet, and the peak and average vertical impulse for each limb. Data feed 312 may include a scrollable interface with additional tracked movement events and data values for the other events. In an implementation, the interface may be filtered to display only events in which lameness was detected. In an implementation, data feed 312 comprises values that correspond to graph 310 and data/or measurement display 304.

The data feed 312 may additionally depict differences in values from average values for the animal. For example, the system may use aggregated data to compute average acceleration values for each limb at a particular gait. During a movement event at the particular gait, the system may compute differences between current acceleration for each limb and average acceleration for that limb. The system may display variation values calculated from average values and current values in order to depict changes in normal behavior. By displaying differences between average values and values for a particular movement event, the system allows a user to determine whether the animal is behaving abnormally.

In an implementation, the data relay device additionally includes a GPS transceiver and/or other positioning identification device. The system may store data identifying location of the animal for different measurement values. Using the measurement values and location data, the system may determine locations where measurements change in the movement of the animal. For example, the system may identify a location where, during a movement event, the difference in vertical impacts for contralateral limbs is greater than a threshold value. The system may display, on a map, an icon indicating the location at which, during the movement event, the difference occurred. The displayed location data may be useful for determining how long to walk a pet. For example, a pet owner may view the map to determine how long the pet owner walked the pet before the pet's limb began to function poorly, thereby allowing the pet owner to adjust walks to match the capabilities of the pet.

Different map displays may demonstrate changes in animal over time, such as for evaluating recovery, responses to medication, and/or changes in energy. For example, a map display may include icons identifying each location where, during a movement event, asymmetry between contralateral limbs surpassed the threshold value. Each icon may additionally indicate a time or date when the movement event took place. Thus, an owner may track changes to the distance an animal is able to walk before movement is impaired.

### Additional Implementations

In an implementation, the data relay device 4 includes one or more force sensors. For example, the data relay device 4 may be placed in a collar for an animal, at a leash attachment site, and/or at another position on the animal. The force sensors may be used to track forces from pushes and pulls originating from a leash attached to the collar. Additionally and/or alternatively, force sensors on a leash may send measurement data to the data relay device 4. Forces from the leash may be used to augment the recording of movement as a portion of the force on one of the limbs would be caused by the pull of the leash.

Additionally and/or alternatively, the data relay device 4 may be configured to detect proximity of the leash to the collar or attachment link. Based on proximity of the leash to the collar and/or data indicating force on the leash, the system may store data identifying the movement event as an event which involves the pet owner. The system may use this data to track how long the owner spends with the pet and/or how often the user takes the pet for walks.

In an implementation, the system is programmed or configured to interact with one or more external devices. For example, the data relay device 4 may connect to one or more devices that monitor a food bowl and/or water bowl. The system may use the data relay device 4 and the food and/or water bowl to compute food and/or water intake for the animal. A measurement device in a food and/or water bowl may initially measure how much food and/or water is in the bowl. For example, a scale on the bowl may weigh food and/or water in the bowl.

A sensor in the food and/or water bowl may be configured to detect close proximity of the data relay device 4 and/or a sensor in the data relay device 4 may be configured to detect close proximity of the food and/or water bowl. The sensors in the food and/or water bowl may send data to the data relay device 4 identifying the weight of the food and/or water in the bowl prior to detection of close proximity of the data relay device 4.

When the food and/or water bowl ceases to be in close proximity to the data relay device 4, the sensors in the bowl may send a second measurement of the weight in the food and/or water bowl to the data relay device 4. Additionally and/or alternatively, one or more processors communicatively coupled to the sensors in the food and/or water bowl may be programmed or configured to compute a difference in weight of the food and/or water and send the difference in weight to the data relay device 4. The system may store a total food and/or water intake for the animal over one or more periods of time based on the differences in weight of the food and/or water in the bowl prior to the detection of close proximity of the animal and after detection of close proximity of the animal.

Monitoring of the food and/or water intake of an animal may be used to detect early symptoms of one or more problems. For example, if an animal's food intake drastically decreases, the system may determine that the animal is not eating and therefore may be sick. As another example, if an animal's water intake drastically increases, the system may determine that the animal is suffering from one or more pathological conditions. Similar systems may be implemented with a litter box to track a cat's frequency of urination and/or to detect other gastrointestinal problems.

In an implementation, a food and/or water bowl uses the detection of the data relay device 4 to apportion food and/or water to the animal. For example, the system may store data identifying an amount of food that is healthy for the animal to eat. When the food bowl detects close proximity of the pet the food bowl may deposit an amount of food based on the data stored by the system indicating a healthy amount of food for the animal. Using these methods, a food bowl may release different amounts of foods to different pets based on detection of proximity of the different pets.

The food bowl may also comprise separate dispensers for separate foods, thereby allowing apportionment of different foods to different pets. Thus, if a first pet is on a diet and can only eat Food A while a second pet only eats Food B, the system may detect close proximity of the data relay device 4 attached to the collar of the first pet and, in response, dispense Food A to the first pet. Food that is not eaten by the pet when the system ceases to detect close proximity of the collar may be automatically covered, such as by a piece of hard plastic.

In an implementation, the food bowls additionally include sensors for weighing a remaining amount of food. The system may be programmed or configured to send a warning when the remaining food has decreased beyond a threshold value. Additionally and/or alternatively, the system may automatically order food from one or more predetermined retailers in response to a determination that the remaining food has decreased beyond the threshold value.

In an implementation, a similar system is implemented for monitoring medication dispensation. For example, a medicine dish may include a bowl or container for the medicine and a scale. The system may determine whether medicine has been removed from the bowl based on a change in weight. For instance, the system may calibrate by weighing a single serving of the medicine and determine that medicine has been given to a pet when the weight of the medicine dish changes by the calibrated weight of the single serving. Medicine dispensation tracking may be used to correlate changes in movement with medicine application. For example, by tracking when the medicine was removed from the dish, the system may determine whether the asymmetry in the animal's movements decreases, increases, or stays the same shortly after medication.

The system may be configured to cause display of reminders on a computing device when medicine has not been removed from the medicine dish when medicine is supposed to be given to the pet. The system may be further configured to remove the reminder when the system identifies a change in weight that indicates that medicine was removed from the bowl.

In an implementation, the system additionally tracks differences based on medicine use. For example, the graphical user interface may include an option for indicating that a medicine was administered to the animal. The system may compare values for vertical impact and/or acceleration after the medicine use with values for vertical impact and/or acceleration prior to the medicine use. By comparing values before and after medicine use, the system is able to use the sensors to record effects of various medicines.

### Machine Learning Overview

A machine learning model is trained using a particular machine learning algorithm. Once trained, input is applied to the machine learning model to make a prediction, which may also be referred to herein as a predicated output or output. Attributes of the input may be referred to as features and the values of the features may be referred to herein as feature values.

A machine learning model includes a model data representation or model artifact. A model artifact comprises parameters values, which may be referred to herein as theta values, and which are applied by a machine learning algorithm to the input to generate a predicted output. Training a machine learning model entails determining the theta values of the model artifact. The structure and organization of the theta values depends on the machine learning algorithm.

In supervised training, training data is used by a supervised training algorithm to train a machine learning model. The training data includes input and a “known” output. In an embodiment, the supervised training algorithm is an iterative procedure. In each iteration, the machine learning algorithm applies the model artifact and the input to generate a predicated output. An error or variance between the predicted output and the known output is calculated using an objective function. In effect, the output of the objective function indicates the accuracy of the machine learning model based on the particular state of the model artifact in the iteration. By applying an optimization algorithm based on the objective function, the theta values of the model artifact are adjusted. An example of an optimization algorithm is gradient descent. The iterations may be repeated until a desired accuracy is achieved or some other criteria is met.

In a software implementation, when a machine learning model is referred to as receiving an input, being executed, and/or generating an output or predication, a computer system process executing a machine learning algorithm applies the model artifact against the input to generate a predicted output. A computer system process executes a machine learning algorithm by executing software configured to cause execution of the algorithm. When a machine learning model is referred to as performing an action, a computer system process executes a machine learning algorithm by executing software configured to cause performance of the action.

Inferencing entails a computer applying the machine learning model to an input such as a feature vector to generate an inference by processing the input and content of the machine learning model in an integrated way. Inferencing is data driven according to data, such as learned coefficients, that the machine learning model contains. Herein, this is referred to as inferencing by the machine learning model that, in practice, is execution by a computer of a machine learning algorithm that processes the machine learning model.

Classes of problems that machine learning (ML) excels at include clustering, classification, regression, anomaly detection, prediction, and dimensionality reduction (i.e. simplification). Examples of machine learning algorithms include decision trees, support vector machines (SVM), Bayesian networks, stochastic algorithms such as genetic algorithms (GA), and connectionist topologies such as artificial neural networks (ANN). Implementations of machine learning may rely on matrices, symbolic models, and hierarchical and/or associative data structures. Parameterized (i.e. configurable) implementations of best of breed machine learning algorithms may be found in open source libraries such as Google's TensorFlow for Python and C++ or Georgia Institute of Technology's MLPack for C++. Shogun is an open source C++ ML library with adapters for several programing languages including C#, Ruby, Lua, Java, MatLab, R, and Python.

### Artificial Neural Networks

An artificial neural network (ANN) is a machine learning model that at a high level models a system of neurons interconnected by directed edges. An overview of neural networks is described within the context of a layered feedforward neural network. Other types of neural networks share characteristics of neural networks described below.

In a layered feed forward network, such as a multilayer perceptron (MLP), each layer comprises a group of neurons. A layered neural network comprises an input layer, an output layer, and one or more intermediate layers referred to hidden layers.

Neurons in the input layer and output layer are referred to as input neurons and output neurons, respectively. A neuron in a hidden layer or output layer may be referred to herein as an activation neuron. An activation neuron is associated with an activation function. The input layer does not contain any activation neuron.

From each neuron in the input layer and a hidden layer, there may be one or more directed edges to an activation neuron in the subsequent hidden layer or output layer. Each edge is associated with a weight. An edge from a neuron to an activation neuron represents input from the neuron to the activation neuron, as adjusted by the weight.

For a given input to a neural network, each neuron in the neural network has an activation value. For an input neuron, the activation value is simply an input value for the input. For an activation neuron, the activation value is the output of the respective activation function of the activation neuron.

Each edge from a particular neuron to an activation neuron represents that the activation value of the particular neuron is an input to the activation neuron, that is, an input to the activation function of the activation neuron, as adjusted by the weight of the edge. Thus, an activation neuron in the subsequent layer represents that the particular neuron's activation value is an input to the activation neuron's activation function, as adjusted by the weight of the edge. An activation neuron can have multiple edges directed to the activation neuron, each edge representing that the activation value from the originating neuron, as adjusted by the weight of the edge, is an input to the activation function of the activation neuron.

Each activation neuron is associated with a bias. To generate the activation value of an activation neuron, the activation function of the neuron is applied to the weighted activation values and the bias.

### Illustrative Data Structures for Neural Network

The artifact of a neural network may comprise matrices of weights and biases. Training a neural network may iteratively adjust the matrices of weights and biases.

For a layered feedforward network, as well as other types of neural networks, the artifact may comprise one or more matrices of edges W. A matrix W represents edges from a layer L−1 to a layer L. Given the number of neurons in layer L−1 and L is N[L−1] and N[L], respectively, the dimensions of matrix W is N[L−1] columns and N[L] rows.

Biases for a particular layer L may also be stored in matrix B having one column with N[L] rows.

The matrices W and B may be stored as a vector or an array in RAM memory, or comma separated set of values in memory. When an artifact is persisted in persistent storage, the matrices W and B may be stored as comma separated values, in compressed and/serialized form, or other suitable persistent form.

A particular input applied to a neural network comprises a value for each input neuron. The particular input may be stored as vector. Training data comprises multiple inputs, each being referred to as sample in a set of samples. Each sample includes a value for each input neuron. A sample may be stored as a vector of input values, while multiple samples may be stored as a matrix, each row in the matrix being a sample.

When an input is applied to a neural network, activation values are generated for the hidden layers and output layer. For each layer, the activation values for may be stored in one column of a matrix A having a row for every neuron in the layer. In a vectorized approach for training, activation values may be stored in a matrix, having a column for every sample in the training data.

Training a neural network requires storing and processing additional matrices. Optimization algorithms generate matrices of derivative values which are used to adjust matrices of weights W and biases B. Generating derivative values may use and require storing matrices of intermediate values generated when computing activation values for each layer.

The number of neurons and/or edges determines the size of matrices needed to implement a neural network. The smaller the number of neurons and edges in a neural network, the smaller matrices and amount of memory needed to store matrices. In addition, a smaller number of neurons and edges reduces the amount of computation needed to apply or train a neural network. Less neurons means less activation values need be computed, and/or less derivative values need be computed during training.

Properties of matrices used to implement a neural network correspond neurons and edges. A cell in a matrix W represents a particular edge from a neuron in layer L−1 to L. An activation neuron represents an activation function for the layer that includes the activation function. An activation neuron in layer L corresponds to a row of weights in a matrix W for the edges between layer L and L−1 and a column of weights in matrix W for edges between layer L and L+1. During execution of a neural network, a neuron also corresponds to one or more activation values stored in matrix A for the layer and generated by an activation function.

An ANN is amenable to vectorization for data parallelism, which may exploit vector hardware such as single instruction multiple data (SIMD), such as with a graphical processing unit (GPU). Matrix partitioning may achieve horizontal scaling such as with symmetric multiprocessing (SMP) such as with a multicore central processing unit (CPU) and or multiple coprocessors such as GPUs. Feed forward computation within an ANN may occur with one step per neural layer. Activation values in one layer are calculated based on weighted propagations of activation values of the previous layer, such that values are calculated for each subsequent layer in sequence, such as with respective iterations of a for loop. Layering imposes sequencing of calculations that is not parallelizable. Thus, network depth (i.e. amount of layers) may cause computational latency. Deep learning entails endowing a multilayer perceptron (MLP) with many layers. Each layer achieves data abstraction, with complicated (i.e. multidimensional as with several inputs) abstractions needing multiple layers that achieve cascaded processing. Reusable matrix based implementations of an ANN and matrix operations for feed forward processing are readily available and parallelizable in neural network libraries such as Google's TensorFlow for Python and C++, OpenNN for C++, and University of Copenhagen's fast artificial neural network (FANN). These libraries also provide model training algorithms such as backpropagation.

### Backpropagation

An ANN's output may be more or less correct. For example, an ANN that recognizes letters may mistake an I as an L because those letters have similar features. Correct output may have particular value(s), while actual output may have somewhat different values. The arithmetic or geometric difference between correct and actual outputs may be measured as error according to a loss function, such that zero represents error free (i.e. completely accurate) behavior. For any edge in any layer, the difference between correct and actual outputs is a delta value.

Backpropagation entails distributing the error backward through the layers of the ANN in varying amounts to all of the connection edges within the ANN. Propagation of error causes adjustments to edge weights, which depends on the gradient of the error at each edge. Gradient of an edge is calculated by multiplying the edge's error delta times the activation value of the upstream neuron. When the gradient is negative, the greater the magnitude of error contributed to the network by an edge, the more the edge's weight should be reduced, which is negative reinforcement. When the gradient is positive, then positive reinforcement entails increasing the weight of an edge whose activation reduced the error. An edge weight is adjusted according to a percentage of the edge's gradient. The steeper is the gradient, the bigger is adjustment. Not all edge weights are adjusted by a same amount. As model training continues with additional input samples, the error of the ANN should decline. Training may cease when the error stabilizes (i.e. ceases to reduce) or vanishes beneath a threshold (i.e. approaches zero). Example mathematical formulae and techniques for feedforward multilayer perceptron (MLP), including matrix operations and backpropagation, are taught in related reference “EXACT CALCULATION OF THE HESSIAN MATRIX FOR THE MULTI-LAYER PERCEPTRON,” by Christopher M. Bishop.

Model training may be supervised or unsupervised. For supervised training, the desired (i.e. correct) output is already known for each example in a training set. The training set is configured in advance by (e.g. a human expert) assigning a categorization label to each example. For example, the training set for optical character recognition may have blurry photographs of individual letters, and an expert may label each photo in advance according to which letter is shown. Error calculation and backpropagation occurs as explained above.

### Autoencoder

Unsupervised model training is more involved because desired outputs need to be discovered during training. Unsupervised training may be easier to adopt because a human expert is not needed to label training examples in advance. Thus, unsupervised training saves human labor. A natural way to achieve unsupervised training is with an autoencoder, which is a kind of ANN. An autoencoder functions as an encoder/decoder (codec) that has two sets of layers. The first set of layers encodes an input example into a condensed code that needs to be learned during model training. The second set of layers decodes the condensed code to regenerate the original input example. Both sets of layers are trained together as one combined ANN. Error is defined as the difference between the original input and the regenerated input as decoded. After sufficient training, the decoder outputs more or less exactly whatever is the original input.

An autoencoder relies on the condensed code as an intermediate format for each input example. It may be counter-intuitive that the intermediate condensed codes do not initially exist and instead emerge only through model training. Unsupervised training may achieve a vocabulary of intermediate encodings based on features and distinctions of unexpected relevance. For example, which examples and which labels are used during supervised training may depend on somewhat unscientific (e.g. anecdotal) or otherwise incomplete understanding of a problem space by a human expert. Whereas, unsupervised training discovers an apt intermediate vocabulary based more or less entirely on statistical tendencies that reliably converge upon optimality with sufficient training due to the internal feedback by regenerated decodings. Techniques for unsupervised training of an autoencoder for anomaly detection based on reconstruction error is taught in non-patent literature (NPL) “VARIATIONAL AUTOENCODER BASED ANOMALY DETECTION USING RECONSTRUCTION PROBABILITY”, Special Lecture on IE. 2015 Dec. 27; 2 (1): 1-18 by Jinwon An et al.

### Principal Component Analysis

Principal component analysis (PCA) provides dimensionality reduction by leveraging and organizing mathematical correlation techniques such as normalization, covariance, eigenvectors, and eigenvalues. PCA incorporates aspects of feature selection by eliminating redundant features. PCA can be used for prediction. PCA can be used in conjunction with other ML algorithms.

### Random Forest

A random forest or random decision forest is an ensemble of learning approaches that construct a collection of randomly generated nodes and decision trees during a training phase. Different decision trees of a forest are constructed to be each randomly restricted to only particular subsets of feature dimensions of the data set, such as with feature bootstrap aggregating (bagging). Therefore, the decision trees gain accuracy as the decision trees grow without being forced to over fit training data as would happen if the decision trees were forced to learn all feature dimensions of the data set. A prediction may be calculated based on a mean (or other integration such as soft max) of the predictions from the different decision trees.

Random forest hyper-parameters may include: number-of-trees-in-the-forest, maximum-number-of-features-considered-for-splitting-a-node, number-of-levels-in-each-decision-tree, minimum-number-of-data-points-on-a-leaf-node, method-for-sampling-data-points, etc.

### Computer Hardware Overview

According to one implementation, the techniques described herein are implemented by one or more special-purpose computing devices. The special-purpose computing devices may be hard-wired to perform the techniques, or may include digital electronic devices such as one or more application-specific integrated circuits (ASICs) or field programmable gate arrays (FPGAs) that are persistently programmed to perform the techniques, or may include one or more general purpose hardware processors programmed to perform the techniques pursuant to program instructions in firmware, memory, other storage, or a combination. Such special-purpose computing devices may also combine custom hard-wired logic, ASICs, or FPGAs with custom programming to accomplish the techniques. The special-purpose computing devices may be desktop computer systems, portable computer systems, handheld devices, networking devices or any other device that incorporates hard-wired and/or program logic to implement the techniques.

For example, FIG. 5 is a block diagram that illustrates a computer system 500 upon which an implementation of the invention may be implemented. Computer system 500 includes a bus 502 or other communication mechanism for communicating information, and a hardware processor 504 coupled with bus 502 for processing information. Hardware processor 504 may be, for example, a general purpose microprocessor.

Computer system 500 also includes a main memory 506, such as a random access memory (RAM) or other dynamic storage device, coupled to bus 502 for storing information and instructions to be executed by processor 504. Main memory 506 also may be used for storing temporary variables or other intermediate information during execution of instructions to be executed by processor 504. Such instructions, when stored in non-transitory storage media accessible to processor 504, render computer system 500 into a special-purpose machine that is customized to perform the operations specified in the instructions.

Computer system 500 further includes a read only memory (ROM) 508 or other static storage device coupled to bus 502 for storing static information and instructions for processor 504. A storage device 510, such as a magnetic disk, optical disk, or solid-state drive is provided and coupled to bus 502 for storing information and instructions.

Computer system 500 may be coupled via bus 502 to a display 512, such as a cathode ray tube (CRT), for displaying information to a computer user. An input device 514, including alphanumeric and other keys, is coupled to bus 502 for communicating information and command selections to processor 504. Another type of user input device is cursor control 516, such as a mouse, a trackball, or cursor direction keys for communicating direction information and command selections to processor 504 and for controlling cursor movement on display 512. This input device typically has two degrees of freedom in two axes, a first axis (e.g., x) and a second axis (e.g., y), that allows the device to specify positions in a plane.

Computer system 500 may implement the techniques described herein using customized hard-wired logic, one or more ASICs or FPGAs, firmware and/or program logic which in combination with the computer system causes or programs computer system 500 to be a special-purpose machine. According to one implementation, the techniques herein are performed by computer system 500 in response to processor 504 executing one or more sequences of one or more instructions contained in main memory 506. Such instructions may be read into main memory 506 from another storage medium, such as storage device 510. Execution of the sequences of instructions contained in main memory 506 causes processor 504 to perform the process steps described herein. In alternative implementations, hard-wired circuitry may be used in place of or in combination with software instructions.

The term “storage media” as used herein refers to any non-transitory media that store data and/or instructions that cause a machine to operate in a specific fashion. Such storage media may comprise non-volatile media and/or volatile media. Non-volatile media includes, for example, optical disks, magnetic disks, or solid-state drives, such as storage device 510. Volatile media includes dynamic memory, such as main memory 506. Common forms of storage media include, for example, a floppy disk, a flexible disk, hard disk, solid-state drive, magnetic tape, or any other magnetic data storage medium, a CD-ROM, any other optical data storage medium, any physical medium with patterns of holes, a RAM, a PROM, and EPROM, a FLASH-EPROM, NVRAM, any other memory chip or cartridge.

Storage media is distinct from but may be used in conjunction with transmission media. Transmission media participates in transferring information between storage media. For example, transmission media includes coaxial cables, copper wire and fiber optics, including the wires that comprise bus 502. Transmission media can also take the form of acoustic or light waves, such as those generated during radio-wave and infra-red data communications.

Various forms of media may be involved in carrying one or more sequences of one or more instructions to processor 504 for execution. For example, the instructions may initially be carried on a magnetic disk or solid-state drive of a remote computer. The remote computer can load the instructions into its dynamic memory and send the instructions over a telephone line using a modem. A modem local to computer system 500 can receive the data on the telephone line and use an infra-red transmitter to convert the data to an infra-red signal. An infra-red detector can receive the data carried in the infra-red signal and appropriate circuitry can place the data on bus 502. Bus 502 carries the data to main memory 506, from which processor 504 retrieves and executes the instructions. The instructions received by main memory 506 may optionally be stored on storage device 510 either before or after execution by processor 504.

Computer system 500 also includes a communication interface 518 coupled to bus 502. Communication interface 518 provides a two-way data communication coupling to a network link 520 that is connected to a local network 522. For example, communication interface 518 may be an integrated services digital network (ISDN) card, cable modem, satellite modem, or a modem to provide a data communication connection to a corresponding type of telephone line. As another example, communication interface 518 may be a local area network (LAN) card to provide a data communication connection to a compatible LAN. Wireless links may also be implemented. In any such implementation, communication interface 518 sends and receives electrical, electromagnetic or optical signals that carry digital data streams representing various types of information.

Network link 520 typically provides data communication through one or more networks to other data devices. For example, network link 520 may provide a connection through local network 522 to a host computer 524 or to data equipment operated by an Internet Service Provider (ISP) 526. ISP 526 in turn provides data communication services through the world wide packet data communication network now commonly referred to as the “Internet” 528. Local network 522 and Internet 528 both use electrical, electromagnetic or optical signals that carry digital data streams. The signals through the various networks and the signals on network link 520 and through communication interface 518, which carry the digital data to and from computer system 500, are example forms of transmission media.

Computer system 500 can send messages and receive data, including program code, through the network(s), network link 520 and communication interface 518. In the Internet example, a server 530 might transmit a requested code for an application program through Internet 528, ISP 526, local network 522 and communication interface 518.

The received code may be executed by processor 504 as it is received, and/or stored in storage device 510, or other non-volatile storage for later execution.

### Cloud Computing

The term “cloud computing” is generally used herein to describe a computing model which enables on-demand access to a shared pool of computing resources, such as computer networks, servers, software applications, and services, and which allows for rapid provisioning and release of resources with minimal management effort or service provider interaction.

A cloud computing environment (sometimes referred to as a cloud environment, or a cloud) can be implemented in a variety of different ways to best suit different requirements. For example, in a public cloud environment, the underlying computing infrastructure is owned by an organization that makes its cloud services available to other organizations or to the general public. In contrast, a private cloud environment is generally intended solely for use by, or within, a single organization. A community cloud is intended to be shared by several organizations within a community; while a hybrid cloud comprises two or more types of cloud (e.g., private, community, or public) that are bound together by data and application portability.

Generally, a cloud computing model enables some of those responsibilities which previously may have been provided by an organization's own information technology department, to instead be delivered as service layers within a cloud environment, for use by consumers (either within or external to the organization, according to the cloud's public/private nature). Depending on the particular implementation, the precise definition of components or features provided by or within each cloud service layer can vary, but common examples include: Software as a Service (SaaS), in which consumers use software applications that are running upon a cloud infrastructure, while a SaaS provider manages or controls the underlying cloud infrastructure and applications. Platform as a Service (PaaS), in which consumers can use software programming languages and development tools supported by a PaaS provider to develop, deploy, and otherwise control their own applications, while the PaaS provider manages or controls other aspects of the cloud environment (i.e., everything below the run-time execution environment). Infrastructure as a Service (IaaS), in which consumers can deploy and run arbitrary software applications, and/or provision processing, storage, networks, and other fundamental computing resources, while an IaaS provider manages or controls the underlying physical cloud infrastructure (i.e., everything below the operating system layer). Database as a Service (DBaaS) in which consumers use a database server or Database Management System that is running upon a cloud infrastructure, while a DbaaS provider manages or controls the underlying cloud infrastructure, applications, and servers, including one or more database servers.

In the foregoing specification, implementations of the invention have been described with reference to numerous specific details that may vary from implementation to implementation. The specification and drawings are, accordingly, to be regarded in an illustrative rather than a restrictive sense. The sole and exclusive indicator of the scope of the invention, and what is intended by the applicants to be the scope of the invention, is the literal and equivalent scope of the set of claims that issue from this application, in the specific form in which such claims issue, including any subsequent correction.

What is claimed is:

1. A computer-implemented method comprising:
receiving, at a wireless data relay device, from a sensor device, first data representative of measurements associated with gait of an animal;
wherein the sensor device is attached to the animal and the sensor device comprises a first wireless transceiver communicatively coupled to one or more first sensors;
determining start times and stop times of movement events associated with the first data;
storing movement event data, wherein the movement event data includes the first data;
using a first machine learning model to generate levels of gait asymmetry for the animal, wherein the movement event data is provided as input for the first machine learning model, and wherein the first machine learning model is trained to predict levels of gait asymmetry using the movement event data;
generating one or more treatment recommendations based on the levels of gait asymmetry for the animal;
displaying, on a client computing device, the one or more treatment recommendations and the levels of gait asymmetry for the animal.

2. The computer-implemented method of claim 1, wherein the first data includes at least one of: kinematic data of the animal, force and pressure data of paws of the animal, GPS data indicating movement of the animal, video data capturing movement of the animal and movement of limbs of the animal, and audio data associated with movement of the animal.

3. The computer-implemented method of claim 1, wherein the sensor device is attached to the animal using one or more straps to attach the sensor device to a limb of the animal.

4. The computer-implemented method of claim 1, wherein the sensor device is affixed to a wearable device worn by the animal.

5. The computer-implemented method of claim 1, wherein the sensor device is part of a footwear element worn on a paw of the animal.

6. The computer-implemented method of claim 1, further comprising:
receiving, at the wireless data relay device, from a second sensor device, second data representative of measurements associated with gait of the animal, wherein the second sensor device is attached to the animal and the second sensor device comprises a second wireless transceiver communicatively coupled to one or more second sensors;
determining second start times and second stop times of second movement events associated with the second data;
wherein storing movement event data comprises storing movement event data that includes the first data and the second data.

7. The computer-implemented method of claim 1, further comprising training the first machine learning model using a training corpus with data representing a plurality of sensor data representing force and pressure data, kinematic data, GPS data, video data, and audio data, for a plurality of animals representing different species of animals, different breeds of each of the species of animals, and different conformations of each of the species and breeds of animals.

8. The computer-implemented method of claim 1, wherein the first machine learning model uses characteristics of the animal to generate the levels of gait asymmetry for the animal, wherein the characteristics of the animal include at least one of: age of the animal, gender of the animal, neutering status of the animal, breed of the animal, weight of the animal, medical history of the animal, observed physical tendencies of the animal, and historical gait observations of the animal.

9. The computer-implemented method of claim 1, wherein generating the one or more treatment recommendations comprises, using a second machine learning model to generate the one or more treatment recommendations for the animal, wherein the second machine learning model is trained to predict the one or more treatment recommendations based on the levels of gait asymmetry for the animal and characteristics of the animal.

10. The computer-implemented method of claim 1, further comprising, displaying, on a graphical user interface on the client computing device, the movement event data including an indication as to whether a limb of the animal is affected by the gait asymmetry.

11. One or more non-transitory computer-readable media storing instructions which, when executed by one or more processors, cause:
receiving, at a wireless data relay device, from a sensor device, first data representative of measurements associated with gait of an animal;
wherein the sensor device is attached to the animal and the sensor device comprises a first wireless transceiver communicatively coupled to one or more first sensors;
determining start times and stop times of movement events associated with the first data;
storing movement event data, wherein the movement event data includes the first data;
using a first machine learning model to generate levels of gait asymmetry for the animal, wherein the movement event data is provided as input for the first machine learning model, and wherein the first machine learning model is trained to predict levels of gait asymmetry using the movement event data;
generating one or more treatment recommendations based on the levels of gait asymmetry for the animal;
displaying, on a client computing device, the one or more treatment recommendations and the levels of gait asymmetry for the animal.

12. The one or more non-transitory computer-readable media of claim 11, wherein the first data includes at least one of: kinematic data of the animal, force and pressure data of paws of the animal, GPS data indicating movement of the animal, video data capturing movement of the animal and movement of limbs of the animal, and audio data associated with movement of the animal.

13. The one or more non-transitory computer-readable media of claim 11, wherein the sensor device is attached to the animal using one or more straps to attach the sensor device to a limb of the animal.

14. The one or more non-transitory computer-readable media of claim 11, wherein the sensor device is affixed to a wearable device worn by the animal.

15. The one or more non-transitory computer-readable media of claim 11, wherein the sensor device is part of a footwear element worn on a paw of the animal.

16. The one or more non-transitory computer-readable media of claim 11, wherein the non-transitory computer-readable media storing further instructions which, when executed by the one or more processors, cause:
receiving, at the wireless data relay device, from a second sensor device, second data representative of measurements associated with gait of the animal, wherein the second sensor device is attached to the animal and the second sensor device comprises a second wireless transceiver communicatively coupled to one or more second sensors;
determining second start times and second stop times of second movement events associated with the second data;
wherein storing movement event data comprises storing movement event data that includes the first data and the second data.

17. The one or more non-transitory computer-readable media of claim 11, wherein the non-transitory computer-readable media storing further instructions which, when executed by the one or more processors, cause, training the first machine learning model using a training corpus with data representing a plurality of sensor data representing force and pressure data, kinematic data, GPS data, video data, and audio data, for a plurality of animals representing different species of animals, different breeds of each of the species of animals, and different conformations of each of the species and breeds of animals.

18. The one or more non-transitory computer-readable media of claim 11, wherein the first machine learning model uses characteristics of the animal to generate the levels of gait asymmetry for the animal, wherein the characteristics of the animal include at least one of: age of the animal, gender of the animal, neutering status of the animal, breed of the animal, weight of the animal, medical history of the animal, observed physical tendencies of the animal, and historical gait observations of the animal.

19. The one or more non-transitory computer-readable media of claim 11, wherein generating the one or more treatment recommendations comprises, using a second machine learning model to generate the one or more treatment recommendations for the animal, wherein the second machine learning model is trained to predict the one or more treatment recommendations based on the levels of gait asymmetry for the animal and characteristics of the animal.

20. The one or more non-transitory computer-readable media of claim 11, wherein the non-transitory computer-readable media storing further instructions which, when executed by the one or more processors, cause, displaying, on a graphical user interface on the client computing device, the movement event data including an indication as to whether a limb of the animal is affected by the gait asymmetry.
